(19) United States
(12) Patent Application Publication
(10) Pub. No.: US 2009/0221066 A1
(43) Pub. Date: Sep. 3, 2009

(54) Anti-Methylated Dna Antibody and Method for Production Thereof

(76) Inventors: Kunio Shiota, Tokyo (JP); Shintaro Yagi, Tokyo (JP); Fumiko Sunaga, Tokyo (JP); Keiji Hirabayashi, Tokyo (JP)

Correspondence Address: BIRCH STEWART KOLASCH & BIRCH, PO BOX 747, FALLS CHURCH, VA, 22040-0747, US

(21) Appl. No.: 12/225,675

(22) PCT Filed: Mar. 27, 2007
(86) PCT No.: PCT/JP2007/056294

§ 371 Date: Sep. 26, 2008

(30) Foreign Application Priority Data
Mar. 28, 2006 (JP) 2006-086948

(51) Int. Cl. C12N 5/22; C07K 16/00

(52) U.S. Cl. 435/345; 530/389.8; 530/388.9

(57) ABSTRACT

The present invention provides a method of preparing an anti-methylated DNA antibody, comprising a step of administering to an animal an oligodeoxyribonucleotide containing a methylated cytosine(s) and an oligodeoxyribonucleotide containing no methylated cytosine. The antibody prepared by this method is capable of detecting a DNA containing a methylated cytosine(s) in a state close to the state of a biological component.

### TECHNICAL FIELD

The present invention relates to an anti-methylated DNA antibody and a method for preparing the antibody. By using the antibody of the present invention, it becomes possible to examine DNA methylation status accurately.

DNA methylation is deeply involved in various events ranging from the development and maintenance of normal cells to the onset and maintenance of diseases such as cancer resulted from occurrence of abnormal cells and maintenance of their characters. Thus, examination of DNA methylation status provides important information for understanding biological phenomena. Further, by examining DNA methylation status, it is possible to diagnose whether cells of interest are normal or abnormal. Further, by regulating DNA methylation, it is possible to restore abnormal cells to normal cells, namely, to cure the cells. Thus, examination of DNA methylation status has a high industrial utility value.

### BACKGROUND ART

Epigenetics refers to “changes in gene function that are inheritable over many cell generations and that do not entail a change (mutation) in nucleotide sequence, or the study of this phenomenon”, and includes the study of a system by which individual cells containing the same genetic information (genome) maintain different characters. The study of epigenetics is one of the important areas of life science which has entered the post-genome age through determination of the entire genomic sequence of human and mouse. The major molecular mechanism of epigenetic gene regulation is DNA methylation and histone modifications.

In vertebrates including mammals, methylation at the 5 position of cytosine (C) in CpG dinucleotides is the only modification observed in genomic DNA and is strongly involved in the regulation of gene activity. Briefly, DNA is heavily methylated and chromosomes are forming heterochromatin (condensed chromatin) where gene expression is severely inhibited (e.g., in the repeat sequences of transposons or in centromeres). While about 70-80% of genomic CpGs are methylated in mammals such as human and mouse, regions with rich CpGs called “CpG islands” are found around promoters or first exons in the gene regions of constitutively expressed genes and have been believed to function as landmarks for gene expression.

However, genome-wide analyses of the DNA methylation status of various tissues or same tissues/cells at different developmental stages focusing on CpG islands revealed that there are tissue/cell-dependent differentially methylated regions (T-DMRs) in tissue/cell-specifically expressed gene regions and that T-DMR methylation status is deeply involved in the expression regulation of tissue/cell-specific genes. It has been also shown that the maintenance and formation of cell/tissue-specific DNA methylation profile consisting of the pattern of the methylation status of T-DMRs over the entire genomic region are important for normal development and cell division.

Through analyses of the methylation status of tumor suppressor genes (such as p16 and p53) using cancer cells and samples from patients, it has been demonstrated that abnormal methylation is one of the important oncogenic mechanisms. Abnormality in methylation is observed not only in a specific gene locus or loci, but global hypomethylation in genomic DNA is observed in cancer cells and cell groups at the pre-oncogenic stage. Thus, it is believed that DNA methylation is deeply involved in oncogenesis and growth of cancer cells after oncogenesis.

Recently, it has become possible to artificially transfer a nucleus of an adult to an egg (nuclear transplantation) and to allow ontogenesis to an adult individual. In clone animals created by such nuclear transplantation, abnormalities such as short life, obesity or renal abnormality occur highly frequently. Genome-wide examination of the methylation status of the genomic DNA of clone animals has found abnormality in DNA methylation status at a plurality of sites. Any of these results strongly suggests that the formation maintenance of DNA methylation profile are important for maintaining cells or living bodies at a normal state and that abnormality in DNA methylation profile is associated with diseases.

Methods of examining DNA methylation status include a method using a restriction enzyme of which the cleavage efficiency varies depending on whether the target DNA is methylated or not. In this method, a tag (or label) is introduced into the restriction site, and after the digested fragments have been fractionated by an appropriate method, the tag (label) is detected. There are other methods: a method in which no tag is introduced but the fractionated patterns of digested fragments are compared using a probe for the fragments; a method in which the fragment is amplified by PCR or the like using the tag at a start point and then the amplified fragment is analyzed; and a method which uses the difference in the sensitivity to chemicals between methylated cytosine and non-methylated cytosine (i.e., methylated cytosine shows resistance to bisulfate reaction), amplifies fragments after bisulfate reaction and analyzes the amplified fragments.

From the viewpoint of genome-wide analysis, any of the above-described methods has a problem of limited analyzable region. For example, in the RLGS method using a restriction enzyme, on the assumption that analyzable gene regions are about 2000 and the number of genes is about 20,000-40,000, only 5 to 10% of the regions can be analyzed even if analyzed evenly. On the other hand, those method combined with PCR are effective when the target of analysis is a specific gene region, but they encounter various problems when a plurality of regions are to be analyzed. In order to achieve comprehensive analysis, still different methods should be combined.

A method is expected in which methylated cytosine is detected directly, or DNA methylation status is examined by discriminating DNA fragments using methylated cytosine as a marker. For example, a method may be given in which DNA fragments are discriminated by the presence/absence of methylated cytosine and the thus discriminated fragments are analyzed. According to this method, it is expected that sequences are less biased compared to the method using a restriction enzyme. With this method, more comprehensive analysis becomes possible. The major problem of this method is how efficiently methylated cytosine can be used as a marker and detected.

Antigen-antibody reaction is widely used as a specific molecule recognition mechanism. Antibodies to methylated cytosine have already been reported. Briefly, the ribose ring of methylcytidine was opened and allowed to covalently bond to a carrier protein (Erlanger and Beiser 1964) to thereby haptenize methylated cytosine. The thus haptenized methylated cytosine was used as an antigen to induce an antibody (Vilpo, Rasi et al. 1984; Reynaud, Bruno et al. 1992; Itoh, Aida et al. 1995). However, this antibody has a big problem of molecule recognition. Since the ribose was opened to be linked to a carrier, the resultant antibody is an antibody that recognizes methylated cytosine, but not an antibody to DNA containing methylated cytosine. Therefore, for detection, it is necessary to expose the methylated cytosine by denaturing DNA by physicochemical techniques using thermal, acid or alkali treatment. The big problem of this antibody is that it is hard to detect methylated cytosines present in double-stranded DNAs in the living body in the original state, namely, in the state of double-stranded DNAs (Non-patent document 1, Non-patent document 2).

[Non-Patent Document 1] Vilpo, J. A., S. Rasi, et al. (1984). “Radioimmunoassay of 5-methyl-2′-deoxycytidine. A method for the quantitation of DNA methylation.” J Immunol Methods 75(2): 241-6.

[Non-Patent Document 2] Hernandez-Blazquez, F. J., M. Habib, et al. (2000). “Evaluation of global DNA hypomethylation in human colon cancer tissues by immunohistochemistry and image analysis.” Gut 47(5): 689-93.

### DISCLOSURE OF THE INVENTION

### Problem for Solution by the Invention

It is an object of the present invention to prepare an antibody to a DNA that contains methylated cytosine-guanine dinucleotides (CpGs) and is in a state close to the state of a biological component.

However, in order to achieve this object, a big problem must be solved. Most of the CpGs present in the genomic DNA of mammals are methylated. In the first place, DNA is a self component, and methylated DNA is also a self component. The immune system in mammals is immuno-tolerant to self components and does not induce production of antibodies to such components. Accordingly, it is difficult to induce the production of an antibody to a DNA containing methylated CpGs as long as a vertebrate is used as a host for immunization.

### Means to Solve the Problem

The present inventors have succeeded in producing an antibody to a DNA containing methylated CpGs using a healthy mammal individual as a host, by improving immunizing antigens, immunizing methods and screening antigens. It was confirmed that, unlike the conventional antibodies to methylated cytosines, the produced antibody strongly responds to undenatured DNA fragments containing methylcytidine in the same manner as it responds to denatured DNA fragments.

Hereinbelow, methods for solving the problem of the present invention will be described. Single-stranded DNA fragments containing unmethylated CpGs show a strong immunoactivation activity (Kreig, Yi et al., 1995). This is a response mediated by TLR9 which specifically recognizes single-stranded DNA fragments containing unmethylated CpGs and which is mainly expressed in pDC cells (Hemmi, Takeuchi et al., 2000). pDC cells are cells whose major role is to present antigen. Innate immune response is induced by TLR9, followed by induction of various cytokines (Akira and Hemmi, 2003).

It has been shown that the immunoactivation activity of DNA fragments containing unmethylated CpGs induces Th1 cytokines and Th2 cytokines in a sequence-dependent manner. Therefore, application of such DNA fragments to the treatment of allergy and immune diseases such as autoimmune diseases which are believed to occur as a result of abnormality in Th1/Th2 balance has been investigated.

On the other hand, attempts to induce conditions of immune diseases have been made paying attention to the fact that administration of a single-stranded DNA containing unmethylated CpGs can vary Th1/Th2 balance. It has been reported that it is possible to induce an antibody to self component by integrating a gene encoding hen egg lysozyme (HEL; having strong antigenicity by itself) into a Tg mouse to make the mouse self-tolerant against HEL antigen and then immunizing the resultant mouse with a DNA fragment containing unmethylated CpGs together with the antigen. Thus, it has been shown that an antibody to self component is inducible, though the report is a very special example using a transgenic mouse, a genetically modified organism (Wand and Krieg, 2004).

Likewise, induction of anti-DNA antibodies in mice immunized with calf thymus DNA as antigen together with a DNA fragment containing unmethylated CpGs has been reported (Pisetsky, Wenk et al. 2001; Tran, Reich et al. 2003). The induced antibodies responded strongly to unmethylated Escherichia coli DNA and to calf thymus DNA where most of the CpGs are believed methylated, with equal intensity. These results indicate that, when a DNA (self component) is used as immunogen in such an immunization method, the induced antibody is an anti-DNA antibody without species specificity On the other hand, the fact that the induced antibody showed the completely equal response to E. coli DNA and calf thymus DNA indicates that antibodies to methylated CpGs have been induced little (Tran, Reich et al. 2003).

In the present invention, in order to obtain an antibody to a DNA containing a methylated cytosine(s), an oligonucleotide containing unmethylated cytosine-guanine dinucleotides is used as an adjuvant for the purpose of allowing the host to recognize the DNA (which is originally a self component) as non-self. At this time, it is obvious that the antibody of interest can not be obtained when a mammalian DNA is used as it is as used in the above reported methods. In order to solve this problem, the present inventors have used an oligonucleotide containing a methylated cytosine(s) as antigen. As a result, the present inventors have succeeded in obtaining the antibody of interest. Thus, the present invention has been achieved.

The present invention provides the following (1) to (9).

(1) An antibody which binds to a double-stranded DNA containing a methylated cytosine(s).
(2) The antibody of (1) above, which does not bind to a DNA containing no methylated cytosine.
(3) The antibody of (1) or (2) above, which is a polyclonal antibody
(4) The antibody of (1) or (2) above, which is a monoclonal antibody
(5) A method of preparing an anti-methylated DNA antibody, comprising a step of administering to an animal an oligodeoxyribonucleotide containing a methylated cytosine(s) and an oligodeoxyribonucleotide containing no methylated cytosine.
(6) The method of (5) above, wherein the oligodeoxyribonucleotide containing a methylated cytosine(s) is bound to a protein.
(7) The method of (5) or (6) above, wherein the animal is a mammal.
(8) The method of any one of (5) to (7) above, comprising a step of collecting serum from the animal administered with oligodeoxyribonucleotides and a step of removing antibodies other than anti-methylated DNA antibody from the collected serum.
(9) The method of any one of (5) to (7) above, comprising a step of collecting splenocytes from the animal administered with oligodeoxyribonucleotides and a step of preparing hybridomas by fusing the collected splenocytes to myeloma cells.

### EFFECT OF THE INVENTION

According to the present invention, it becomes possible to detect a DNA containing a methylated cytosine(s) in a state close to the state of a biological component. As a result, it becomes possible to examine the status of DNA methylation more accurately and in more detail.

### BRIEF DESCRIPTION OF THE DRAWINGS

FIG. 1 shows the chemical bond between methylated ODN and a carrier protein schematically. R1 represents the carrier protein.

FIG. 2 shows the reactivities of domestic rabbit immune sera to methylated ODN.

FIG. 3 shows the reactivity of domestic rabbit 4 serum to SssI methylase-treated E. coli DNA.

FIG. 4 shows the reactivities of domestic rabbit 4 immune serum and a conventional anti-methylated cytosine antibody to heat treated DNA (single-stranded DNA) and non-heat treated DNA (double-stranded DNA). Filled columns show the reactivities of domestic rabbit 4 immune serum. Open columns show the reactivities of the conventional anti-methylated cytosine antibody.

FIG. 5 shows the reactivities of mouse sera (dilution: 1000) to non-heat treated DNA. Filled columns show the reactivities to mouse liver DNA. Open columns show the reactivities to E. coli (JM110) DNA.

FIG. 6 shows the reactivities of mouse monoclonal antibodies to mouse DNA and E. coli DNA. Filled columns show the reactivities to mouse DNA. Open columns show the reactivities to E. coli DNA.

FIG. 7 shows the effect of thermal treatment of mouse genomic DNA upon reactivity to a mouse monoclonal antibody. Filled columns show the reactivities to heat treated DNA. Open columns show the reactivities to non-heat treated DNA.

### BEST MODE FOR CARRYING OUT THE INVENTION

Hereinbelow, the present invention will be described in detail.

The antibody of the present invention is an antibody which binds to a double-stranded DNA containing a methylated cytosine(s).

As described earlier, antibodies to methylated cytosine have been known (see, for example, Non-Patent Document 1 and Non-Patent Document 2). However, these antibodies can bind to methylated cytosine only when the methylated cytosine has been exposed by denaturing DNA. An antibody capable of binding to a DNA containing a methylated cytosine(s) in the state of double-stranded DNA (without denaturing) at a practical level has been created for the first time by the present inventors.

The antibody of the present invention may be any antibody as long as it binds to a DNA containing a methylated cytosine(s). Preferably, the antibody of the present invention is an antibody which does not bind to a DNA containing no methylated cytosine. Also preferable is an antibody that binds to a double-stranded DNA containing a methylated cytosine(s) with intensity almost equal to the intensity with which it binds to a single-stranded DNA containing a methylated cytosine(s). The expression “binds . . . with intensity almost equal to the intensity with which it binds to a single-stranded DNA containing a methylated cytosine(s)” used herein means, for example, showing 50% or more binding relative to the binding to a single-stranded DNA.

The antibody of the present invention includes both polyclonal antibody and monoclonal antibody, and humanized antibody.

The method of preparation of the anti-methylated DNA antibody of the present invention is characterized by comprising a step of administering to an animal an oligodeoxyribonucleotide containing a methylated cytosine(s) (hereinafter, referred to as “methylated ODN”) and an oligodeoxyribonucleotide containing no methylated cytosine (hereinafter, referred to as “adjuvant ODN”). It should be noted here that the above-described antibody of the present invention is included in the “anti-methylated DNA antibody” used in this paragraph.

Methylated ODN may be administered as it is. However, for enhancing its immunogenicity, methylated ODN is preferably administered in a state of being linked to a protein, wherein the linkage does not change the structure of the methylated oligonucleotide. As the protein, any protein with immunogenicity may be used. For example, bovine serum albumin, KLH (keyhole limpet hemocyanin), ovalbumin, and the like may be enumerated.

The length of methylated ODN is not particularly limited. The length is preferably in the range from 10 to 50 based, more preferably in the range from 20 to 30 bases.

The nucleotide sequence of methylated ODN is not particularly limited as long as the sequence contains methylated cytosine-guanine dinucleotides and does not contain non-methylated cytosine-guanine dinucleotides. The number of methylated cytosines contained in methylated ODN is not particularly limited. Preferably, a plurality of methylated cytosines are contained.

Adjuvant ODN may be a natural oligodeoxyribonucleotide, but preferably a phosphorothioated oligodeoxyribonucleotide is used.

The length of adjuvant ODN is not particularly limited as long as the adjuvant ODN has immunoactivation activity. The length is preferably in the range from 10 to 50 based, more preferably in the range from 20 to 30 bases.

An adjuvant other than adjuvant ODN may be administered together with methylated ODN. Preferably, an adjuvant that does not act through toll-like receptor-9 (TLR-9) is used. Examples of preferable adjuvants include, but are not limited to, incomplete Freund's adjuvant, Alum and oil.

The type of animal to which methylated ODN, etc. are to be administered is not particularly limited. Although domestic rabbit and mouse are used to obtain antibodies in the Examples, at least animals such as monkey, pig, sheep, goat and rat may be used because the adjuvant activity of oligonucleotides containing CpG is confirmed in these animals (Kandimalla, Bhagat et al. 2003; Nichani, Kaushik et al. 2004). Alternatively, animals to be administered are not limited to these animals; mammals in general may be the animals to be administered.

The dose of methylated ODN, etc. may be determined depending on the type of animal to be administered and is not particularly limited. When administered to domestic rabbit, the dose of methylated ODN per administration may be 1 μg to 1 mg, and the dose of adjuvant ODN per administration may be 10 μl to 1 ml.

Methylated ODN, etc. may be administered 2 to 10 times at intervals of 1 to 8 weeks.

The method of preparation of an anti-methylated DNA antibody of the present invention comprises a step of administering to an animal methylated ODN and adjuvant ODN. As long as the method comprises this step, other steps are not particularly limited.

For example, a polyclonal antibody to methylated DNA may be prepared by collecting serum from an animal administered with methylated ODN, etc. and then removing antibodies other than anti-methylated DNA antibody from the serum. The antibodies to be removed here include antibodies to unmethylated DNA, contaminants contained at the time of immunization, e.g., antibodies to the protein (carrier protein) linked to methylated ODN, and the like. As a method for removing these antibodies, affinity chromatography using a resin to which unmethylated DNA is bound may be given as one example. In this method, antibodies to unmethylated DNA may be removed by recovering non-bound fractions. Likewise, affinity chromatography using a resin to which the carrier protein is bound may be used. In this method, antibodies to the carrier may be removed by recovering non-bound fractions.

Alternatively, a monoclonal antibody to methylated DNA may be prepared by collecting splenocytes from an animal administered with methylated ODN, etc. and fusing the splenocytes to myeloma cells to prepare hybridomas. Those cell clones which produce antibodies to DNA containing a methylated cytosine(s) do not respond to DNA containing no methylated cytosine. Therefore, such cell clones may be easily selected using, as an indicator, the reactivity to DNA containing a methylated cytosine(s).

Further, it is possible to isolate the cDNA of an anti-methylated DNA antibody by collecting the mRNA from splenocytes of the animal administered with methylated ODN, etc., and making selections using recombinant DNA techniques and also using, as an indicator, the non-reactivity to DNA containing no methylated cytosine and the reactivity to DNA containing a methylated cytosine(s). From the thus isolated cDNA, it is possible to create a recombinant antibody (Holliger and Hudson 2005; Hoogenboom 2005). Further, it is also possible to establish according to conventional methods a humanized monoclonal antibody which responds to DNA containing a methylated cytosine(s) (Lonberg 2005).

### EXAMPLES

Hereinbelow, the present invention will be described in more detail with reference to the following Examples.

### Example 1

### Synthesis of Oligodeoxyribonucleotides and Preparation of DNAs

As adjuvant ODN, phosphorothioated ODNs with the sequences described below were used. They were synthesized by the phosphoamidite method and purified with HPLC column. The synthesis was entrusted to Sigma Genosys.

As methylated ODN, an ODN with the sequence described below was used. This ODN was synthesized by the phosphoamidite method and purified with HPLC column. The synthesis was entrusted to Sigma Genosys.

TABLE 1

| | |
|---|---|
| ODN2007 | tcgtcgttgtcgttttgtcgtT |
| ODN1826 | tccatgacgttcctgacgtT |
| Methylated ODN | 2TOGTOGTTTTOGGOGGCOGCC |

In the Table, “O” represents methylated cytosine. Small letters represent phosphorothioated sequences.

The preparation of DNAs from E. coli and mouse liver was performed using Wizard SV Genome DNA kit (Promega) and according to the protocol recommended by the manufacturer. DNAs digested with HeaIII were used for determining antibody titers. DNA concentrations were calculated based on the absorbance at 260 nm.

### Example 2

### Preparation of Immunogen (Methylated ODN-BSA Conjugate)

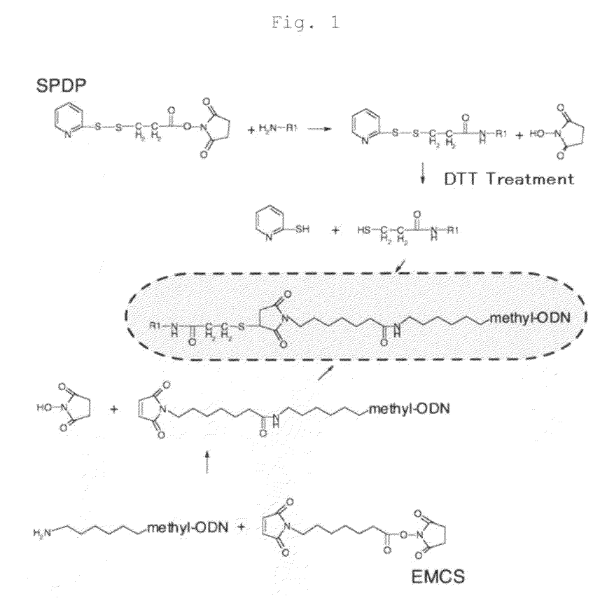

An outline of synthesis reactions for methylated ODN-BSA conjugate is shown in FIG. 1. Briefly, 25 μl of 20 mM SPDP (N-succinimidyl 3-(2-pyridyldithio) propionate; Pierce) was added to 1 ml of 10 mg/ml [phosphate buffer (20 mM sodium phosphate, 150 mM NaCl, 1 mM EDTA, 0.02% NaN₃, pH 7.5)] bovine serum albumin (BSA) solution and incubated at room temperature for 2 hours. The reaction solution was applied to NAP-10 Column (Amersham Bioscience) to remove unreacted SPDP and eluted with 1.5 ml of phosphate buffer. Subsequently, 750 μl of labeled BSA prepared to give a concentration of 24 mg/ml [sodium acetate buffer [100 mM sodium acetate, 100 mM 100 mM NaCl, (pH 4.5), 1 mM DTT] was added thereto and incubated at room temperature for 1 hour. Using NAP-25 Column (Amersham Bioscience), the buffer was exchanged with 3.5 ml of phosphate buffer and a part thereof was used for the conjugate with methylated ODN described later (BSA-SPDP).

To 180 μl of methylated ODN diluted to give a concentration of 5 mg/ml [phosphate buffer (20 mM sodium phosphate, 150 mM NaCl, pH 7.0)], 20 PI of 10 mM EMCS(N-[ε-maleimidocaproyloxy]succinimide ester; Dojindo) was added and incubated at room temperature for 2 hours. The reaction solution was applied to NAP-10 Column (Amersham Bioscience) to remove unreacted EMCS and eluted with 1.5 ml of phosphate buffer (pH 7.0). Subsequently, 231 μl of BSA-SPDP was added thereto and incubated at room temperature for 1 hour. Finally, after concentration with Centricut20 (Cosmo Bio), the buffer was exchanged with 1.5 ml of phosphate buffer (pH 7.0) in Nap-10 column to thereby obtain methylated ODN-BSA conjugate (hereinafter, referred to as “BSAmODN”). The concentration was determined with BCA Protein Assay Reagent (Pierce).

### Example 3

### Immunization of Domestic Rabbits

20 μg of BSAmODN and 20 μg of adjuvant ODN(ODN2007) were emulsified together Freund's incomplete adjuvant. Domestic rabbits (white colored, Japanese species) were immunized subcutaneously with the resultant emulsion. After 5 times of immunization at intervals of 2 weeks, whole blood was collected from each rabbit and serum was isolated therefrom according to conventional methods.

### Example 4

### Antibody Titer Measuring Antibody (Methylated ODN-OVA Conjugate)

To 1 ml of ovalbumin (OVA) at a concentration of 10 g/ml [phosphate buffer (20 mM sodium phosphate, 150 mM NaCl, 1 mM EDTA, 0.02% NaN₃, pH 7.5)], 25 μl of 20 mM SPDP (N-succinimidyl 3-(2-pyridyldithio) propionate; Pierce) was added and incubated at room temperature for 2 hours. After removal of unreacted SPDP with NAP-10 Column (Amersham Bioscience) and elution with 1.5 ml of phosphate buffer, 750 μl of labeled OVA solution prepared to given a concentration of 24 mg/ml [sodium acetate buffer [100 mM sodium acetate, 100 mM 100 mM NaCl, (pH 4.5), 1 mM DTT] was added to the eluate and incubated at room temperature for 1 hour. Using NAP-25 Column (Amersham Bioscience), the buffer was exchanged with 3.5 ml of phosphate buffer and a part thereof was used for the conjugate with methylated ODN described later (OVA-SPDP).

To 180 μl of methylated ODN diluted to give a concentration of 5 mg/ml [phosphate buffer (20 mM sodium phosphate, 150 mM NaCl, pH 7.0)], 20 μl of 10 mM EMCS(N-[ε-maleimidocaproyloxy]succinimide ester) was added and incubated at room temperature for 2 hours. The reaction solution was applied to NAP-10 Column to remove unreacted EMCS and eluted with 1.5 ml of phosphate buffer (pH 7.0). Subsequently, 231 μl of OVA-SPDP was added thereto and incubated at room temperature for 1 hour. Finally, after concentration with Centricut20 (Cosmo Bio), the buffer was exchanged with 1.5 ml of phosphate buffer (pH 7.0) in Nap-10 column to thereby obtain methylated ODN-OVA conjugate (hereinafter, referred to as “OVAmODN”). The concentration was determined with BCA Protein Assay Reagent (Pierce).

### Example 5

### Determination of Antibody Titers by ELISA

Methylated ODN-OVA conjugate, methylated ODN-BSA conjugate and individual proteins not linked to methylated ODN were diluted with PBS to give a concentration of 2.5 μg/ml. Diluted antigen was placed in Maxisorb multimodule plates (Nunc) (100 μl/well) and allowed to stand still at room temperature for 2 hours. After removal of the diluent, a blocking solution (0.5% casein, TBS) was added to the plates (100 μl/well) and allowed to stand still at room temperature for 4 hours to thereby immobilize the antigen on the plate.

For immobilization of DNA, DNA diluted with sterilized water to give a concentration of 3 μg/ml was placed in Cova-link multimodule plates (Nunc) (50 μl/well). To each well, 37.5 μl of 100 mM 1-methyl-imidazole (pH 7.0) and 12.5 μl of EDC (1-ethyl-3-[3-dimethylaminopropyl]carbodiimide hydrochloride) diluted to 10 mM in 1-methyl-imidazole (pH 7.0) solution were added and kept at 50° C. for 4 hours to overnight to thereby allow covalent binding of the DNA. Each well was washed with TBS containing 0.1% Tween20 (T-TBS). Then, a blocking solution (0.5% casein, T-TBS) was added at 100 μl/well and allowed to stand still at 4° C. overnight.

To each well, 100 μl of rabbit serum diluted with a sample diluent (0.1% casein, TBS) was added and allowed to stand still for 1 hour. After washing each well with TBS containing 0.1% Tween20, 100 μl of horseradish peroxidase-labeled anti-rabbit IgG antibody was added and allowed to stand still for 1 hour. After washing each well with TBS containing 0.1% Tween20, 100 μl of OPD color former (Sigma) was added and incubated for 30 minutes. Subsequently, 2N sulfate solution was added and absorbance at 490 nm was measured.

### Example 6

### Confirmation of Anti-Methylated Cytosine DNA Antibody

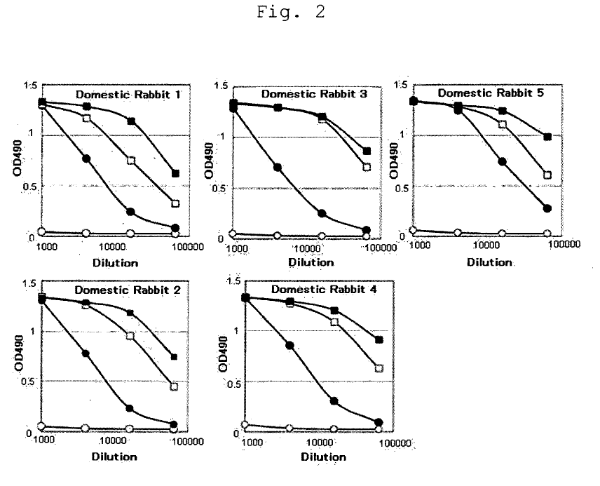

Sera were collected from 5 domestic rabbits which were immunized with BSAmODN in the same manner in the Example. The reactivities of the antibodies in the sera were confirmed by ELISA. Sera before immunization showed no reactivity to antigens BSAmODN and BSA. On week 5 (results not shown) and 8 of the immunization, every serum revealed strong reactivity (FIG. 2). However, their reactivities varied; they showed stronger reactivity to BSAmODN. Similar results were obtained against OVAmODN. Although there was individual difference, sera before immunization showed no reactivity to OVAmODN but on week 5 (results not shown) and 8 of the immunization, every serum revealed strong reactivity (FIG. 2). On the other hand, sera did not respond to OVA not linked to methylated ODN. These results indicate that antibodies to methylated ODN have been produced by immunization.

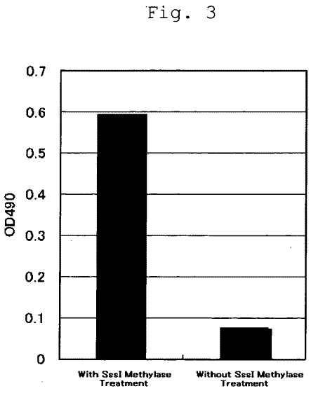

The following observations were made on the serum of domestic rabbit 4 which showed a high antibody titer in the above examination. Antibodies to DNA in which only C in CpG is methylated were confirmed by the examination described below. SssI DNA methylase is an enzyme which specifically methylates only C in CpG As a double-stranded DNA in which no CpGs are methylated, a DNA fragment prepared from E. coli JM110 was used. This DNA fragment (substrate) was treated with SssI methylase. Subsequently, responses to the treated fragment and SssI methylase-untreated fragment were compared by ELISA (FIG. 3). A remarkable rise in response to SssI methylase-treated fragment was confirmed. From this result, it was confirmed that antibodies to a DNA fragment in which C in CpG is methylated have been produced.

### Example 7

### Difference from the Conventional Anti-Methylated Cytosine Antibody in Reactivity

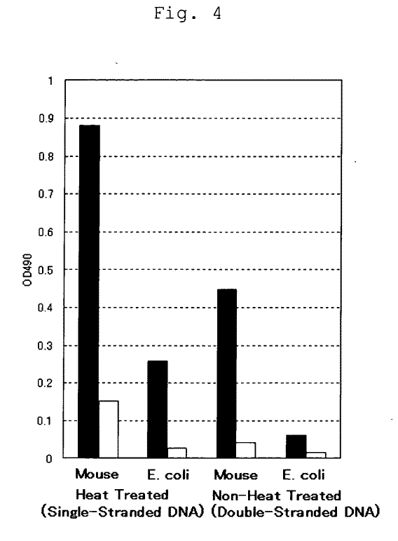

Most of cytosines in CpG dinucleotides in the genomic DNA of mammals are methylated. On the other hand, cytosines in E. coli genomic DNA are not methylated. Reactivities to a DNA purified from mouse liver and to a DNA purified from E. coli JM110 strain were confirmed by ELISA (FIG. 4). The immune serum obtained from domestic rabbit 4 is showing stronger response to mouse liver DNA than E. coli JM110 strain, which strongly support that antibodies to methylated DNA have been produced. On the other hand, the conventional anti-methylated cytosine antibody responds little to non-heat treated fragments.

Subsequently, the serum and the conventional antibody were examined from the presence or absence of difference in reactivity resulted from thermal denaturation of DNA. Since the conventional anti-methylated cytosine antibody strongly responds to heat denatured mouse liver DNA (single-stranded) but does not respond to heat denatured E. coli DNA (single-stranded), it was confirmed that this antibody recognizes heat denatured methylated DNA (single-stranded) (FIG. 4). However, the reactivity of this antibody was remarkably lowered to non-heat denatured mouse liver DNA (double-stranded) (FIG. 4). On the other hand, the immune serum obtained from domestic rabbit 4 showed strong reactivity regardless of the presence or absence of thermal denaturation (FIG. 4). These results indicate that the immune serum obtained from domestic rabbit 4 contains antibodies totally different from the conventional anti-methylated cytosine.

### Example 8

### Fractionation of Anti-Methylated DNA Antibody

The antiserum obtained by the immunization method described in Example 2 responds to both E. coli DNA and BSA which is not linked to mODN. Therefore, it is clear that this antiserum contains anti-DNA antibodies and anti-BSA antibodies. These antibodies were removed by affinity chromatography.

A BSA-linked column was prepared by allowing BSA to covalently bond to Hi-trap NHS activated column according to the method recommended by the manufacturer. An E. coli DNA-linked column was prepared by allowing DNA to covalently bond to CNBr activated sepharose according to the method recommended by the manufacturer. The removal of anti-BSA antibodies and anti-DNA antibodies was confirmed by examining the reactivities to BSA and E. coli DNA by ELISA. Collecting non-bound fractions from affinity chromatography, it was confirmed that anti-BSA antibody titer was lowered to 1/91 and that anti-DNA antibody titer was decreased to an undetectable level. Through these steps, an antibody to methylated DNA could be purified.

TABLE 2

| | Quantity of | | Anti-Mouse DNA | Anti-BSA | Anti-E. coli DNA |
|---|---|---|---|---|---|
| | Total Protein | | Antibody Titer | Antibody Titer | Antibody Titer |
| Serum | 58 | mg | 4,000 | 64,000 | 1,000 |
| After fractionation with | 7 | mg | 1,080 | 288 | 331 |
| BSA-linked column | | | | | |
| After fractionation with | 5.3 | mg | 1,140 | 70 | Not |
| DNA-linked column | | | | | detectable |

### Example 9

### Production of Anti-Methylated DNA Antibody in Mouse

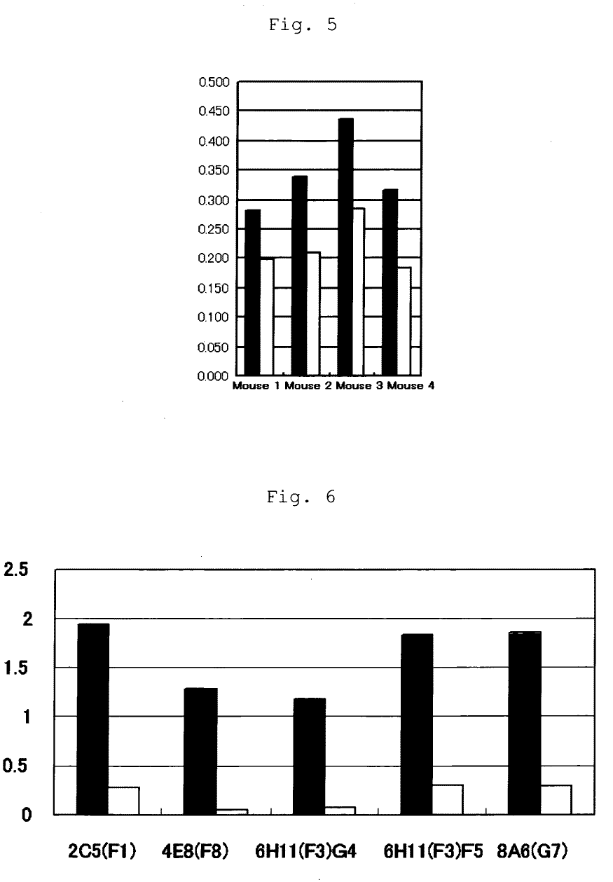

Mice were immunized by the method described in Example 2. The reactivities of the resultant antisera were confirmed by the method described in Example 6. As adjuvant ODN, ODN1826 was used. The antisera collected from the immunized mice responded more strongly to mouse liver DNA, though responded to E. coli DNA also. Thus, it was confirmed that anti-methylated DNA antibodies have been produced in mice as in domestic rabbits (FIG. 5).

### Example 10

### Establishment of Anti-Methylated Oligonucleotide Mouse Monoclonal Antibody Producing Clones

Three days before cell fusion, splenocytes were removed from each mouse administered with BSAmODN and centrifuged in PBS (1,200 rpm, 5 minutes, room temperature) for washing. The resultant pellet containing splenocytes was suspended in RPMI1640 medium (serum free, 10 ml). Myeloma cells (SP2 strain) cultured in advance for cell fusion were harvested and suspended in RPMI1640 medium. The above-described splenocytes and myeloma cells were mixed at a ratio of 5:1 and centrifuged at 1,200 rpm for 5 minutes to thereby obtain a cell precipitate. (splenocytes:myeloma cells)=(5:1)=(6.25×10⁷ cells:1.25×10⁷ cells). 50% PEG was added to the cell precipitate to achieve cell fusion. The resultant cells were centrifuged at 1,200 rpm for 5 minutes and the supernatant was discarded. 50 ml of 10% FBS RPMI1640 medium (supplemented with 10% BM-Condimed H1/HAT) was added thereto for suspending cells. The resultant cell suspension was plated on five 96-well culture plates per mouse at 100 μl/well. Five days thereafter, 10% FBS RPMI1640 medium (without 10% BM-Condimed H1/HAT) was added at 100 μl/well. From the 96-well culture plate (containing cells), culture supernatant was dispensed with an octapipette into antigen (BSAmODN)-immobilized 96-well ELISA plates (100 μl/well). Those clones which produce culture supernatant presenting positive reaction in ELISA were screened for. The culture supernatants presenting positive reaction were further subjected to ELISA using a mouse DNA-immobilized plate and an E. coli DNA-immobilized plate, and those clones which strongly respond to mouse DNA but do not strongly respond to E. coli DNA were selected. Positive cells were plated on 96-well culture plates at 4 cells/well by the limiting dilution method. Five days thereafter, 10% FBS RPMI1640 medium (without BM-Condimed H1/HAT) was dispensed to each plate at 100 μl/well. When it became possible to confirm colonies by eyes and the medium presented a light yellow color after several days' culture, screening was performed. Positive cell-containing wells were selected in the same manner as described above and subjected to secondary cloning by the limiting dilution method (1 cell/well). Then, culture supernatants containing anti-methylated ODN monoclonal antibodies were screened again. The thus selected clones were cultured for another several days. When cells grew to some extent, secondary cloning was performed.

Five cell samples were taken from the positive wells obtained by the screening after the secondary cloning, and plated on 24-well culture plates containing 10% FBS-RPMI1640 medium (supplemented with 2.5% BM-Condimed H1/HAT) (1 ml of medium/well). When cells grew to some extent after several days' culture, each cell sample was transferred into 25 cm culture flasks and grown in 5 ml of 10% FBS-RPMI1640 medium (supplemented with 2.5% BM-Condimed H1/HAT). Then, the cell became capable of culturing in 10% FBS-RPMI1640 medium and came close to confluence, each cell sample was stocked frozen in 2 cryotubes (Cellbanker). By the above-described method, 5 monoclonal antibody producing clones of 2C5(F1), 4E8(F8), 6H11(F3)G4, 6H11(F3)F5 and 8A6(G7) were established.

### Example 11

### Confirmation of the Reactivities of the Monoclonal Antibodies

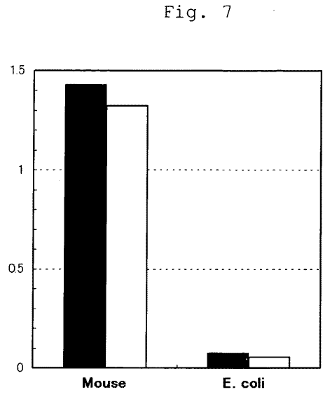

The reactivities of the monoclonal antibodies obtained from the established cell clones to non-heat denatured mouse DNA and E. coli DNA were examined by ELISA on culture supernatants from individual monoclonal antibody producing cell clones. As shown in FIG. 6, it was confirmed that every monoclonal antibody presented strong reactivity to non-heat denatured mouse DNA but responded little to E. coli DNA.

Difference in reactivity caused by thermal denaturation was examined on 6H11(F3)G4 monoclonal antibody. As shown in FIG. 7, the monoclonal antibody showed equal reactivity to non-heat denatured mouse genomic DNA and to heat denatured mouse genomic DNA.

### REFERENCES

Akira, S, and H. Hemmi (2003). “Recognition of pathogen-associated molecular patterns by TLR family” Immunol Lett 85: 85-95.
Erlanger, B. F. and S. M. Beiser (1964). “Antibodies Specific For Ribonucleosides And Ribonucleotides And Their Reaction With Dna.” Proc Natl Acad Sci USA 52: 68-74.
Hattori, N., K. Nishino, et al. (2004). “Epigenetic control of mouse Oct-4 gene expression in embryonic stem cells and trophoblast stem cells.” J Biol Chem 279(17): 17063-9.
Hemmi, H., O. Takeuchi, et al. (2000). “A Toll-like receptor recognizes bacterial DNA.” Nature 408(6813): 740-5.
Hernandez-Blazquez, F. J., M. Habib, et al. (2000). “Evaluation of global DNA hypomethylation in human colon cancer tissues by immunohistochemistry and image analysis.” Gut 47(5): 689-93.
Holliger, P. and P. J. Hudson (2005). “Engineered antibody fragments and the rise of single domains.” Nat Biotechnol 23(9): 1126-36.
Hoogenboom, H. R (2005). “Selecting and screening recombinant antibody libraries.” Nat Biotechnol 23(9): 1105-16.
Itoh, K., S. Aida, et al. (1995). “Immunochemical detection of urinary 5-methyl-2′-deoxycytidine as a potential biologic marker for leukemia.” Clin Chim Acta 234(1-2): 37-45.
Kandimalla, E. R., L. Bhagat, et al. (2003). “A dinucleotide motif in oligonucleotides shows potent immunomodulatory activity and overrides species-specific recognition observed with CpG motif.” Proc Natl Acad Sci USA 100: 14303-8.
Krieg, A. M., A. K. Yi, et al. (1995). “CpG motifs in bacterial DNA trigger direct B-cell activation.” Nature 374(6522): 546-9.
Lonberg, N. (2005). “Human antibodies from transgenic animals.” Nat Biotechnol 23(9): 1117-25.
Nichani, A. K., R. S. Kaushik, et al. (2004). “CpG oligodeoxynucleotide induction of antiviral effector molecules in sheep.” Cell Immunol 227: 24-37.
Pisetsky, D. S., K. S. Wenk, et al. (2001). “The role of cpg sequences in the induction of anti-DNA antibodies.” Clin Immunol 100: 157-63.
Reynaud, C., C. Bruno, et al. (1992). “Monitoring of urinary excretion of modified nucleosides in cancer patients using a set of six monoclonal antibodies.” Cancer Lett 61(3): 255-62.
Tran, T. T., A. Reich, et al. (2003). “Specificity and immunochemical properties of anti-DNA antibodies induced in normal mice by immunization with mammalian DNA with a CpG oligonucleotide as adjuvant.” Clin Immunol 109: 278-87.
Vilpo, J. A., S. Rasi, et al. (1984). “Radioimmunoassay of 5-methyl-2′-deoxycytidine. A method for the quantitation of DNA methylation.” J Immunol Methods 75(2): 241-6.
Wang, Y and A. M. Krieg (2004). “Induction of autoantibody production but not autoimmune disease in HEL transgenic mice vaccinated with HEL in combination with CpG or control oligodeoxynucleotides.” Vaccine 22: 2641-50.

The present specification encompasses the contents of disclosed in the specification and/or drawings of Japanese Patent Application No. 2006-086948 based on which the present patent application claims priority. All publications, patents and patent applications cited herein are incorporated herein by reference in their entirety.

1. An antibody which binds to a double-stranded DNA containing a methylated cytosine(s).

2. The antibody according to claim 1, which does not bind to a DNA containing no methylated cytosine.

3. The antibody according to claim 1 or 2, which is a polyclonal antibody.

4. The antibody according to claim 1 or 2, which is a monoclonal antibody.

5. A method of preparing an anti-methylated DNA antibody, comprising a step of administering to an animal an oligodeoxyribonucleotide containing a methylated cytosine(s) and an oligodeoxyribonucleotide containing no methylated cytosine.

6. The method according to claim 5, wherein the oligodeoxyribonucleotide containing a methylated cytosine(s) is bound to a protein.

7. The method according to claim 5 or 6, wherein the animal is a mammal.

8. The method according to claim 5, comprising a step of collecting serum from the animal administered with oligodeoxyribonucleotides and a step of removing antibodies other than anti-methylated DNA antibody from the collected serum.

9. The method according to claim 5, comprising a step of collecting splenocytes from the animal administered with oligodeoxyribonucleotides and a step of preparing hybridomas by fusing the collected splenocytes to myeloma cells.


SEQUENCE LISTING (3 sequences)

SEQ ID NO: 1 - DNA, 22 bp, Artificial Sequence
  Description of Artificial Sequence synthetic DNA
tcgtcgttgtcgttttgtcgtt

SEQ ID NO: 2 - DNA, 20 bp, Artificial Sequence
  Description of Artificial Sequence synthetic DNA
tccatgacgttcctgacgtt

SEQ ID NO: 3 - DNA, 21 bp, Artificial Sequence
  Description of Artificial Sequence synthetic DNA
tcgtcgttttcggcggccgcc